(19) United States
(12) Patent Application Publication
(10) Pub. No.: US 2009/0253601 A1
(43) Pub. Date: Oct. 8, 2009

(54) FLOATING COMBI-BAR AND MIXTURE FOR PRODUCING SAME

(76) Inventors: Tee Yong Tan, Singapore (SG); Norbert Braun, Singapore (SG); Jason Hock Chye Tan, Singapore (SG); Wolfgang Neugebauer, Singapore (SG)

Correspondence Address: CONNOLLY BOVE LODGE & HUTZ LLP, 1875 EYE STREET, N.W., SUITE 1100, WASHINGTON, DC, 20006, US

(21) Appl. No.: 12/089,991

(22) PCT Filed: Oct. 12, 2005
(86) PCT No.: PCT/EP2005/055203

§ 371 Date: Aug. 11, 2008

(51) Int. Cl. A61K 8/02

(52) U.S. Cl. 510/145

(57) ABSTRACT

Described is a mixture for producing a combi-bar floating in water at 20° C. (floating combi-bar), the mixture comprising or (essentially) consisting of:
v) 20-50 wt. %, preferably 25-40 wt. %, of one or more non-soap surfactants, preferably solid non-soap surfactants,
vi) 25-60 wt. %, preferably 35-55 wt. %, of one or more free C₁₄-C₁₈ fatty acids,
vii) 2-40 wt. %, preferably 5-25 wt. %, of one or more metal C₈-C₂₄ fatty acid soaps, and
viii) 0-20 wt. %, preferably 2-20 wt. %, of further ingredients,
   said mixture containing less than 10 wt. % of water as a further ingredient.

Further described are methods for producing a combi-bar and a combi-bar itself.

### TECHNICAL FIELD

The present invention relates to a mixture for producing a combi-bar floating in water at 20° C. (and other temperatures), i.e. having a density below 0.99821 g/mL at 20° C. Such combi-bar hereinafter are also designated as “floating combi-bar”. The present invention also relates to a method for producing a floating combi-bar and a combi-bar itself.

The combi-bar of the present invention does not require an aeration step during preparation (during the manufacturing process) to attain buoyancy. Upon hardening at room temperature, the combi-bar of the present invention is able to float when placed in water.

A combi-bar, by definition, is a bar comprising a combination of soap and non-soap surfactants.

### BACKGROUND & PRIOR ART

The appeal of a floating soap-containing bar (and in particular a floating combi-bar) is that the user need not search for a missing bar in the bottom of a tub filled with soapy water. The prior art comprises floating soap bars in which air is incorporated and is responsible for the bars to float on water. A classic example is the Ivory soap of Procter & Gamble where air is mechanically whipped into the soap mass to achieve buoyancy of the final soap bar.

U.S. Pat. No. 2,295,594 discloses a process for obtaining a floating soap comprising mechanical air entrapment through whipping and extrusion of soap in a condition of pasty cohesiveness such that air in finely divided bubble form can be incorporated. The bars are allowed to cool and harden after extrusion. The soap bar compositions do not contain any non-soap detergent active.

U.S. Pat. No. 5,972,860 discloses an aerated detergent bar essentially incorporating inorganic salts and/or polyols and non-ionic surfactants wherein the air is in the fine bubble form and entrapped in the formulation before casting.

U.S. Pat. No. 5,194,172 discloses an aerated freezer bar comprising fatty acid soap, sucrose, and hydrophobic material selected from waxes, and water.

U.S. Pat. No. 5,219,487 discloses an aerated freezer soap bar comprising fatty acid soap, free fatty acid, salt, and water.

U.S. Pat. No. 6,770,606 discloses a method whereby the air is incorporated into the bar after the soap mass is cast. Air is entrained in the bar after it has been dehydrated in the oven.

U.S. Pat. No. 5,602,088 discloses a method of extruding a soap billet with a hollow recess that is pneumatically sealed off when stamped into shape. Air trapped within the hollow recess will allow the soap to float on water.

The prior arts manufacturing process comprise an air-incorporating step and corresponding equipment. It was an object of the present invention to provide a floating soap-containing bar which can be produced without an aeration step. Additionally, corresponding mixtures and production methods (methods of manufacture) should be provided.

Regarding the aspect “mixture”, the present invention concerns a mixture for producing a combi-bar floating in water at 20° C. and other temperatures (floating combi-bar), the mixture comprising or (essentially) consisting of:
i) 20-50 wt. %, preferably 25-40 wt. %, of one or more non-soap surfactants, preferably solid non-soap surfactants,
ii) 25-60 wt. %, preferably 35-55 wt. %, of one or more free C₁₄-C₁₈ fatty acids,
iii) 2-40 wt. %, preferably 5-25 wt. %, of one or more metal C₈-C₂₄ fatty acid soaps, and
iv) 0-20 wt. %, preferably 2-20 wt. %, of further ingredients,
   said mixture containing less than 10 wt. % of water, preferably less than 6 wt. % of water, as a further ingredient.

Particularly preferred is a mixture according to the present invention which comprises or (essentially) consists of:
i) 25-40 wt. % of one or more non-soap surfactants, preferably solid non-soap surfactants,
ii) 35-55 wt. % of one or more free C₁₄-C₁₈ fatty acids,
iii) 5-25 wt. % of one or more metal C₈-C₂₄ fatty acid soaps, and
iv) 2-15 wt. % of further ingredients,
   said mixture containing less than 6 wt. % of water.

It has surprisingly been found that casting a mixture according to the present invention, i.e. a mixture comprising specific amounts of non-soap ingredients, free fatty acids and soap results in a composition which can be given the shape of a combi-bar which in turn is solid at room temperature (20° C.), has the ability to float on water (at 20° C. and other temperatures), wherein the method of production does not need any aeration or air entrapment steps. Using the mixture of the present invention in the production of floating combi-bars results in a shortened manufacturing process and a reduction of capital investment, as air entrapment and/or aeration equipment is not necessary.

The mixture of the present invention differs from prior art mixtures for producing floating soap bars as such prior art mixtures are predominately based on soap and only to a minor extent comprise non-soap surfactants and/or fatty acids. According to the present invention a considerable amount of non-soap surfactants (20-50 wt. %) and of free C₁₄-C₁₈ fatty acids (25-60 wt. %) is used. In contrast thereto, U.S. Pat. No. 5,219,487 discloses the use of 70-80% soap with a maximum fatty acid content of 5% while U.S. Pat. No. 5,972,860 discloses a composition with 20%-50% soap and a lower amount of non-soap surfactant at 1-15%.

Throughout these specification, unless indicated otherwise, all percentages and ratios given are by weight and all conditions and properties (e.g. state of aggregation) refer to 20° C.

Preferably, in mixtures of the present invention the total amount of components i), ii), and iii) is at least 80 wt. %, and is preferably in the range of 85 to 98 wt. %.

A further aspect of the present invention concerns a method for producing a combi-bar floating in water at 20° C., comprising the following steps:

a) providing a mixture according to the present invention (as disclosed above and, preferably, as described in more detail below),
b) heating said mixture to a temperature in the range of 70-90° C. to obtain a pourable melt,
c) casting or pouring said melt into a mould,
d) cooling said melt in said mould so that the melt solidifies, and
e) removing the resulting combi-bar from said mould,
wherein in the process no or only so little air or other gas is incorporated into the mixture or melt that the resulting combi-bar contains less than 2 vol % of air or other gas.

A further aspect of the present invention concerns a combi-bar floating in water at 20° C. (floating combi-bar) and preferably other temperatures, comprising a mixture according to the present invention, the combi-bar containing less than 2 vol % of air or other gas. Such combi-bar is obtainable by a method according to the present invention, i.e. a method using a mixture according to the present invention.

In a preferred method of the present invention, for manufacturing floating combi-bars the non-soaps surfactants (component i)) are dissolved in the molten free fatty acids (component ii)) and thereto the fatty acid soap (component iii)) as well as the further ingredients (component iv)), if present are added. The resulting molten mass comprising components i), ii), iii), and optionally component iv) is subsequently poured or cast into a mould (step c) and cooled in said mould to room temperature (20° C.). To aid the cooling process the moulds may be placed in a tray of cold water. When the melt has solidified (hardened), the resulting combi-bar is removed from the mould. No or only so little air or other gas is incorporated into the mixture or melt that the resulting combi-bar contains less than 2 vol % of air or other gas.

The mixtures of the present invention and the corresponding combi-bars of the present invention contain less than 10 wt. % of water, preferably less than 6 wt. % of water, for the following reasons:
When incorporating an amount of more than 10 wt. % of water, the mixture before casting attains a very high viscosity that is impractical for casting into moulds. However, the hardened mass still floats when placed in water.
When incorporating an amount in a range of from 6 wt. % to 10 wt. % of water into the formulation the mixture before casting attains a high viscosity. Although the mixture can be cast into moulds, the resulting combi-bars sometimes have a rough surface which lacks smoothness. The resulting combi-bars float when placed in water.
Best results are obtained when less than 6 wt. % of water are incorporated into the mixtures and combi-bars of the present invention.

The mixtures and combi-bars of the present invention preferably contain not more than 5 wt. % of diols or polyols, e.g. glycerol (glycerine). In a preferred embodiment the total content of diols and polyols is not more than 3 wt. %.

The mixtures and combi-bars of the present invention are preferably free of reducing sugars, in particular free of sucrose.

The combi-bars of the present invention may be of any size or shape. Their shape for example may be round, oval, rectangular, quadratic, spherical, ellipsoidal, cuboid, helical or irregularly shaped, e.g. in the form of figures like crocodiles, cats, horses, ducks, roses, sunflowers, shells, fish, whales, dolphins, ships, stars, starfish.

Hereinafter, the terms “coconut”, “cocamido” or “cocoyl” or the like which are used in connection with soap or non-soap surfactants refer to materials having an approximate carbon chain length distribution of: 4-9% C₈; 4-9% C₁₀, 45-55% C₁₂, 15-21% C₁₄, 8-13% C₁₆, 7-14% C₁₈, and about 5-7% oleic and 1-3% linoleic (the first six fatty acids being saturated, oleic and linoleic acid being unsaturated).

Hereinafter, the term “tallow” refers to a mixture of soaps having an approximate chain length distribution of: 2-4% C₁₄; 23-32% C₁₈; 14-25% C₁₈; 2-4% palmitoleic; 36-48% oleic and 2-8% linoleic (the first three fatty acids being saturated, paimitoleic, oleic and linoleic acid being unsaturated).

Preferred Components i), ii), iii), and iv) are now described in more detail:

### Non-Soap Surfactants (Component i)):

The non-soap surfactants of component i) according to the present invention are substances that are preferably solid at 20° C. Non-soap surfactants such as C₈-C₂₂-alkyl sulphates (e.g. sodium laureth sulfate, sodium lauryl sulfate), C₈-C₂₂-alkyl sulphoacetates, sulphosuccinates (in particular di-C₈-C₂₂-alkyl-sulphosuccinates, e.g. sodium dioctyl sulphosuccinates), monoglyceride sulphates, acyl isethionates, betaines, glyceryl ether sulphonates, C₈-C₂₂-alkylsulphonates, ether sulphonates, acylsulphonates, alkylacyl sulphonates and C₈-C₁₆-alkyl glucosides are preferred.

Preferably mild non-soap surfactants, anionic or amphoteric, are used. Preferred surfactants are sodium cocoyl isethionate, cocoamidopropyl betaine and sodium lauryl sulfosuccinate. These surfactants can commercially be obtained from (product names or trademarks): Sodium Cocoyl Isethionate [Clariant (Hostapon SCI-65), Akzo Nobel (Elfan AT 84)], Cocamidopropyl Betaine [Goldschmidt (Tego Betaine CKD)], Sodium Lauryl Sulfosuccinate [Goldschmidt (Rewopol SB F 12 P)]. C₈-C₁₆-alkyl glucosides, e.g. Decyl Glucoside or Lauryl Glucoside are available as Plantaren® 1200 UP or Plantaren® 2000 UP from Henkel.

Free C₁₄-C₁₈ Fatty Acids (Component ii))

The fatty acids of component ii) according to the present invention are, preferably saturated, C₁₄-C₁₈ fatty acids, and can aid in lowering the pH and add hardness to the floating combi-bar. Preferred C₁₄-C₁₆ fatty acids are saturated, i.e. “hardened”, and can be obtained by hydrogenation of unsaturated C₁₄-C₁₈ fatty acids. Most preferred fatty acids are selected from myristic, palmitic and stearic acids.

The free C₁₄-C₁₈ fatty acids improve the quantity and quality of the lathering characteristics of bars prepared in accordance with the present invention, the advantage of free C₁₄-C₁₈ fatty acids being in tending to provide a lather of desirable stability and having small air bubbles so as to provide a rich or creamy lather. Free C₁₄-C₁₈ fatty acids also provide an emollient effect, which tends to soften the skin or otherwise improve feel-on-skin characteristics and scavenge any excess alkalinity.

Preferred fatty acids can commercially be obtained from (product names or trademarks): Myristic Acid [Uniqema (Prifrac 2942), Akzo Nobel (Kortacid 1499), Cognis (Edenor C14 98-100)], Mixtures of Palmitic/Stearic Acid [Uniqema (Pristerene 4900), Akzo Nobel (Kortacid PH 10), Cognis (Edenor ST 1 MY, Edenor L2SM, Edenor FHTi)], Palmitic Acid [Akzo Nobel (Kortacid 1698), Cognis (Edenor C16 98-100)], Stearic Acid [Akzo Nobel (Kortacid 1898), Cognis (Edenor C18 98-100)].

Metal C₈-C₂₄ Fatty Acid Soaps (Component iii))

Component iii) according to the present invention comprises one or more metal soaps of C₈-C₂₄ fatty acids. Preferred are alkali metal, in particular sodium, potassium, or lithium, magnesium, aluminum, calcium salts of fatty acids as component iii) of the floating combi-bar of the present invention. Particularly preferred are alkali metal, preferably sodium or potassium soaps, most preferably sodium soaps, of fatty acids to increase the hardness of the bar.

The soaps (component iii)) of the present invention preferably contain from 8 to 18 carbon atoms. Commercial soaps preferred herein are generally based upon mixtures of fatty acids obtained from various natural sources. Coconut oil, for example, is a material, which has found considerable use in high-quality soap compositions. Similarly, tallow is a useful source of high-quality soaps. Other suitable sources include palm kernel oil and babassu kernel oil, olive oil and synthetic fatty acids simulating, for example, tallow. Particularly useful herein are the sodium or potassium salts of the mixtures of fatty acids derived from coconut oil, palm oil, palm kernel oil and tallow, e.g. sodium or potassium tallow and coconut soap. These soaps and soap mixtures are preferred from the standpoint of ready availability and ease of processing, and in view of their desirably optimum physical and performance characteristics.

Suitable commercially available fatty acids soaps (component iii)) are available from Uniqema and include Prisavon 9220 (Soap from Palm/Palm Kernel fatty acids), Prisavon 9240 (Soap from Palm/Coconut fatty acids), and Prisavon 9250 (Soap from Tallow/Coconut fatty acids).

Further Ingredients (Component iv))

All ingredients of a mixture or combi-bar of the present invention which do not qualify as one of components i), ii) or iii) are considered a further ingredient. E.g., water maybe further ingredient of a mixture or combi-bar of the present invention, up to a concentration of 10 wt. %.

Further ingredients which can be present in the mixtures and floating combi-bars according to the invention can give rise to additional effects. Examples which may be mentioned are: preservatives, abrasives, anti-acne agents, agents against skin aging, anti-bacterial agents, anticellulitis agents, antidandruff agents, anti-inflammatory agents, antiirritants, irritation suppressants, antimicrobial agents, antioxidants, astringents, perspiration suppressants, antiseptics, antistatics, binders, buffers, carrier materials, chelating agents, cell stimulants, cleaning agents, treatment preparations, depiliatories, deodorisers, antiperspirants, softeners, emollients, emulsifiers, enzymes, fibres, film-forming agents, fixatives, foaming agents, foam stabilisers, antifoams, foam boosters, fungicides, gelling agents, gel-forming agents, haircare agents, hair-shaping agents, hair-smoothing agents, moisture donors, humectants, moisturisers, bleaching agents, strengthening agents, stain removers, optical brighteners, impregnating agents, soil repellents, friction-reducing agents, lubricants, opacifiers, plasticisers, polish, brighteners, polymers, powders, proteins, fat restorers, abrasive agents, silicones, skin-calming agents, skin-cleansing agents, skin care agents, skin-healing agents, skin-lightening agents, skin-protecting agents, skin-softening agents, cooling agents, skin-cooling agents, warming agents, skin-warming agents, stabilisers, UV-absorbers, UV-filters, detergents, soft rinses, suspending agents, skin-tanning agents, thickeners, vitamins, oils, waxes, fats, phospholipids, mono- or polyunsaturated fatty acids, α-hydroxy acids, polyhydroxy fatty acids, liquefiers, dyes, color-protection agents, pigments, anti-corrosives, aromas, flavorings, fragrances, perfumes, or essential oils other customary constituents of a cosmetic or dermatological formulation, such as alcohols, polyols, electrolytes, organic solvents or silicone derivatives.

The fragrance compounds or perfumes for use in the mixtures and combi-bars of this invention may be any fragrance compounds or perfumes known to the art. It is a characteristic of this invention that an unusually broad range of fragrance compounds or perfumes may be used. Examples include digeranyl succinate, dineryl succinate, geranyl neryl succinate, geranyl phenylacetate, neryl phenylacetate, geranyl laurate, neryl laurate, di(citronellyl) maleate, dinonanyl maleate, diphenoxyethyl maleate, di(3,7-dimethyl-1-octanyl) succinate, di(cyclohexylethyl) maleate, di(phenylethyl) adipate, 7-acetyl-1,2,3,4,5,6,7,8-octahydro-1,1,6,7-tetramethyl naphthalene, ionone methyl, ionone gamma methyl, methyl cedrylone, methyl dihydrojasmonate, methyl 1,6,10-trimethyl-2,5,9-cyclododecatrien-1-yl ketone, 7-acetyl-1,1,3,4,4,6-hexamethyl tetralin, 4-acetyl-6-tert-butyl-1,1-dimethyl indane, para-hydroxy-phenyl-butanone, benzophenone, methyl beta-naphthyl ketone, 6-acetyl-1,1,2,3,3,5 hexamethyl indane, 5-acetyl-3-isopropyl-1,1,2,6-tetramethyl indane, 1-dodecanal, 4-(4-hydroxy-4-methylpentyl)-3-cyclohexene-1-carboxaldehyde, 7-hydroxy-3,7-dimethyl octanal, 10-undecen-1-al, isohexenyl cyclohexyl carboxaldehyde, formyl tricyclodecane, condensation products of hydroxycitronellal and methyl anthranilate, condensation products of hydroxycitronellal and indol, condensation products of phenyl acetaldehyde and indol, 2-methyl-3-(para-tert-butylphenyl)-propionaldehyde, ethyl vanillin, vanillin, heliotropin, hexyl cinnamic aldehyde, amyl cinnamic aldehyde, 2-methyl-2-(para-iso-propylphenyl)propionaldehyde, coumarin, decalactone gamma, cyclopentadecanolide, 16-hydroxy-9-hexadecenoic acid lactone, cyclohexadecanone, 8-cyclohexadecenone, 1,3,4,6,7,8-hexahydro-4,6,6,7,8,8-hexamethylcyclo-penta-gamma-2-benzopyrane, beta-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, para-(tert-butyl)cyclohexyl acetate, essential oils, resinoids, and resins from a variety of sources including but not limited to orange oil, lemon oil, patchouli, Peru balsam, Olibanum resinoid, styrax, labdanum resin, nutmeg, cassia oil, benzoin resin, coriander, lavandin, and lavender, phenyl ethyl alcohol, terpineol, linalool, linalyl acetate, geraniol, nerol, 2-(1,1-dimethylethyl)cyclohexanol acetate, benzyl acetate, orange terpenes, eugenol, diethylphthalate, and combinations thereof.

The fragrance compounds or perfumes are generally present in a total amount of 0.05 to 5 wt. %, preferably of 0.1 to 2.5 wt. %, and particularly preferentially of 0.2 to 1.5 wt. %, based on the total weight of the floating soap combi-bar.

The fragrance compounds or perfumes can be added in liquid form, undiluted or diluted with a solvent. Suitable solvents for this purpose are, for example, ethanol, isopropanol, diethylene glycol monoethyl ether, glycerol (glycerine), propylene glycol, 1,2-butylene glycol, dipropylene glycol, diethyl phthalate, triethyl citrate, isopropyl myristate, etc.

Furthermore, the fragrance compounds or perfumes for the mixtures and combi-bars according to the invention can be adsorbed on a carrier which serves both for fine dispersion of the fragrance compounds or perfumes in the product and also for controlled release during use. Such carriers can be porous inorganic materials, such as light sulphate, silica gels, zeolites, gypsums, clays, clay granules, etc. or organic materials such as woods and cellulose-based substances.

The mixtures and combi-bars according to the invention can contain plant parts and plant extracts. Examples which may be mentioned are arnica, aloe, usnea, stinging nettle, ginseng, henna, camomile, marigold, rosemary, sage, horsetail or thyme.

Furthermore, cosmetic oils which can be applied dermally can be incorporated into the mixtures and floating combi-bars of the present invention, such as, for example, neutral oils of the Miglyol 812 type, apricot kernel oil, avocado oil, babussu oil, cottonseed oil, borage oil, thistle oil, peanut oil, gamma-oryzanol, rosehip kernel oil, hemp oil, hazelnut oil, currant seed oil, jojoba oil, cherry stone oil, salmon oil, linseed oil, maize germ oil, macadamia nut oil, almond oil, evening primrose oil, mink oil, olive oil, pecan nut oil, peach kernel oil, pistachio kernel oil, rapeseed oil, rice germ oil, casyor oil, safflower oil, sesame oil, soybean oil, sunflower oil, tea tree oil, grapeseed oil or wheatgerm oil.

The mixtures and combi-bars of the present invention can contain UV-absorbers (UV-filters), such as, for example, Neo Heliopane® (Symrise) to protect against discoloration of the soap or protect against solar irradiation on the skin. The UV-absorbers can be UV-A and/or UV-B-absorbers.

Suitable UV-absorbers are, for example, organic UV-absorbers from the class of 4-aminobenzoic acid and derivatives, salicylic acid derivatives, benzophenone derivatives, dibenzoylmethane derivatives, diphenyl acrylates, 3-imidazol-4-yl-acrylic acid and esters thereof, benzofuran derivatives, benzylidene malonate derivatives, polymeric UV-absorbers, containing one or more silicoorganic radicals, cinnamic acid derivatives, camphor derivatives, trianilino-s-triazine derivatives, 2-hydroxyphenylbenzotriazole derivatives, 2-phenylbenzimidazole-5-sulphonic acid and salts thereof, methyl anthranilate, benzotriazole derivatives.

Preferred UV-absorbers are: 4-aminobenzoic acid, 2-ethylhexyl 4-dimethylaminobenzoate, 4-aminobenzoic acid ethyl ester, homomethyl salicylat, 2-ethylhexyl salicylate, 5-methyl-2-(1-methylethyl)cyclohexyl 2-aminobenzoate, ethylhexyl 4-methoxycinnamate, isoamyl 4-methoxycinnamate, 2-ethylhexyl-2-cyano-3,3-diphenylacrylate, 2-phenylbenzimidazole-5-sulfonic acid and salts thereof, 3-(4′-trimethylammonium)benzylidenbornan-2-one methyl sulfate, 3-(4′-sulfo)benzylidenebornan-2-one and the salts thereof, terephthalylidene-dibornansulfonic acid and salts thereof, 4-t-butyl-4′-methoxydibenzoylmethane, 2-hydroxy-4-methoxybenzophenone, 2-hydroxy-4-methoxybenzophenon-5-sulfonic acid and its sodium salt, 3-(4′-methylbenzylidene) camphor, 3-benzylidenecamphor, 2,4,6-trianilino-(p-carbo-2′-ethylhexyl-1′-oxy)-1,3,5-triazine, phenylene-1,4-bis-(2-benzimidazyl)3,3′-5,5′-tetrasulfonic acid and the salts thereof, 2,2′-(1,4-phenylene)bis-(1H-benzimidazole-4,6-disulfonic acid, mono sodium salt), N-[(2 and 4)-[2-(oxoborn-3-ylidene)methyl]benzyl]-acrylamide-polymer, 2-(2H-benzotriazole-2-yl)-4-methyl-6-(2-methyl-3-(1,3,3,3-tetra methyl-1-(trimethylsilyl)-oxy)-disiloxyanyl)propyl)-phenol, 4,4′-[(6-[4-(1,1-Ddimethyl)aminocarbonyl)phenylamino]-1,3,5-triazine-2,4-diyl)dimino]-bis-(benzoic acid-2-ethylhexylester), 2,2′-methylene-bis-(6-(2H-benztriazole-2-yl)-4-(1,1,3,3-tetramethylbutyl)-phenol, 2,4-bis-[4-(2-ethylhexyloxy)-2-hydroxyphenyl]-1,3,5-triazine, benzylidenemalonate-polysiloxane, 4,4′,4-(1,3,5-triazine-2,4,6-triyl-triimino)-tris-benzoic acid tris(2-ethylhexyl ester), 2,4-bis-[{(4-(2-ethyl-hexyloxy)-2-hydroxy}phenyl]-6-(4-methoxyphenyl)-1,3,5-triazine.

The total amount of UV-absorbers, if at all present, preferably is in the range of 0.1 to 6 wt. %, based on the total weight of the combi-bar or mixture.

Moreover, it is possible to use particulate UV-filters or inorganic pigments, which optionally can be rendered hydrophobic, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures thereof.

The incorporation of cooling agents into the mixtures and combi-bars of the present invention is also advantageous. Examples of suitable cooling agents are: 1-menthol, menthone-glycerol acetal, menthyl lactate, substituted menthyl-3-carboxamides (e.g. menthyl-3-carboxylic acid N-ethylamide), 2-isopropyl-N,2,3-trimethylbutanamide, substituted cyclohexanecarboxamides, 3-menthoxypropane-1,2-diol, 2-hydroxyethyl menthylcarbonate, 2-hydroxypropyl menthylcarbonate, N-acetylglycine menthyl ester, menthyl hydroxycarboxylates (e.g. menthyl 3-hydroxybutyrate), monomenthyl succinate, 2-mercaptocyclodecanone, menthyl-2-pyrrolidin-5-one carboxylate.

Substances with a warming effect can advantageously be incorporated into the mixtures and combi-bars according to the invention. Examples are: capsaicin; dihydrocapsaicin; gingerol; paradol; shogaol; piperin; paprika powder, chilli pepper powder, extracts of paprika, extracts of pepper; extracts of chilli pepper; extracts of root ginger; extracts of Aframomum melgueta, extracts of spilanthesacmella; extracts from Kaempferia galanga; extracts of Alpinia galanga, carboxylic acid N-vanillylamides, in particular nonanoic acid N-vanillylamide; 2-nonenoic acid amides, in particular 2-nonenoic acid N-isobutylamide; 2-nonenoic acid N-4-hydroxy-3-methoxyphenylamide; alkyl ethers of 4-hydroxy-3-methoxybenzyl alcohol, in particular 4-hydroxy-3-methoxybenzyl n-butyl ether; alkyl ethers of 3-hydroxy-4-methoxybenzyl alcohol; alkyl ethers of 3,4-dimethoxybenzyl alcohol; alkyl ethers of 3-ethoxy-4-hydroxybenzyl alcohol; alkyl ethers of 3,4-methylenedioxybenzyl alcohol; (4-hydroxy-3-methoxyphenyl)acetamides, in particular (4-hydroxy-3-methoxyphenyl)acetic acid N-n-octylamide; nicotinaldehyde; methyl nicotinate; propyl nicotinate, 2-butoxyethyl nicotinate, benzyl nicotinate and 1-acetoxychavicol.

The mixtures and combi-bars according to the invention can contain antimicrobially active ingredients or preservatives. These can be quaternary monoammonium salts, such as cocoalkylbenzyldimethylammonium chloride, (C₁₂-C₁₄)alkylbenzyldimethylammonium chloride, 1-(3-chloroallyl)-3,5,7-triaza-1-azoniaadamantane chloride (Dowicil®), cocoalkyldichlorobenzyldimethylammonium chloride, tetradecylbenzyldimethylammonium chloride, didecyldimethylammonium chloride, dioctyldimethylammonium chloride, myristyltrimethylammonium bromide, cetyltrimethylammonium bromide, monoquaternary heterocyclic amine salts, such as, for example, laurylpyridinium chloride cetylpyridinium chloride, (C₁₂-C₁₄)alkylbenzylimidazolium chloride, triphenylphosphonium salts, such as, for example, myristyltriphenylphosphonium bromide, halogenated biocides, for example hypochlorites or sodium dichloroisocyanurates, phenolic biocides, such as, for example, phenol and its derivatives, phenol ethers, monoalkylphenols, polyalkylphenols, arylphenols, o-phenylphenol, p-tert-butylphenol, 6-n-amyl-m-cresol, 4,4′-diamidino-α,ω-diphenoxypropane diisethionate (propamidine isethionate), 4,4′-diamidino-α,ω-diphenoxyhexane diisethionate (hexamidine isethionate), alkyl- and/or aryl-chloro- or -bromophenols, such as, for example, o-benzyl-p-chlorophenol, resorcinol and its derivatives, such as, for example, resorcinol monoacetate, cresols, p-chloro-m-xylene, dichloro-m-xylene, 4-chloro-m-cresol, halogenated diphenyl ethers, such as, for example, 2′,4,4′-trichloro-2-hydroxydiphenyl ether (Triclosan) or 2,2′-dihydroxy-5,5′-dibromodiphenyl ether, chlorophenesine (p-chlorophenyl glycerol ether), bisphenol compounds, bis(2-hydroxy-3,5-dichlorophenyl) sulphide, bis(2-hydroxy-5-chlorobenzyl) sulphide, halogenated carbanilides, such as, for example, 3,4,4′-trichlorocarbanilide, pyrithione, in particular the sodium and zinc compounds, Octopirox®, Nuosept®, Nuosept C®, dimethyldimethylolhydantoin (DMDM, Glydant®), 3-butyl-2-iodopropinyl carbamate, Glydant Plus®, 3-isothiazolone compounds, isothiazolinones (“Kathon”), methylisothiazolinone, methylchloroisothiazolinone, diazolidinylurea (Germall II®), imidazolidinylurea (Abiol®, Unicide U-13®, Germall 115®), benzyl alcohol, bicyclic polymethoxyoxazolidinones (e.g. Nuosept®C), 2-bromo-2-nitropropane-1,3-diol (Bronopol®), iodopropenylbutyl carbamate (Polyphase P100®), chloroacetamide, methanamine, 1,2-dibromo-2,4-dicyanobutane (Tektamer®), 5-bromo-5-nitro-1,3-dioxane (Bronidox®), phenethyl alcohol, o-phenylphenol, sodium o-phenylphenol, sodium hydroxymethylglycinate (Suttocide A®), dimethoxane, thimerosal, dichlorobenzyl alcohol, captan, chlorophenesine, dichlorophene, chlorobutanol and glyceryl laurate, glutardialdehyde, parabens (e.g. methylparaben, ethylparaben, propylparaben and butylparaben), phenoxyethanol, sorbitol, dibromodicyanobutane, imidazolidinylureas (“Germall”), organic acids (e.g. benzoic acid, sorbic acid, salicylic acid), and esters thereof, glycols, e.g. propylene glycol, 1,2-di-hydroxyalkanes), ethoxylated, propoxylated or mixed ethoxylated/propoxylated fatty alcohols, fatty acids and fatty acid esters of chain lengths C₈ to C₂₂ with 1 to 250 ethylene glycol ether (EO) and/or propylene glycol ether (PO) units and combinations thereof.

In the mixtures and combi-bar of the present invention it is also possible to use so-called “natural” antibacterial active compounds; these are mostly essential oils. Typical assential oils having an antibacterial action are, for example, oils obtained from aniseed, lemon, orange, rosemary, wintergreen, thyme, lavender, hops, citronella, wheat, lemon-grass, cedarwood, cinnamon, geranium, sandalwood, violet, eucalyptus, peppermint, gum benzoin, basil, fennel, menthol and Ocmea origanum, Hydastis carradensis, Berberidaceae daceae, Ratanhiae or Curcuma longa.

Important antimicrobially active substances that can be found in essential oils and can be integrated in the mixtures and combi-bars of the present invention are, for example, anethole, catechol, camphene, carvacrol, eugenol, eucalyptol, ferulic acid, farnesol, hinokitiol (sic), tropolone, limonene, menthol, methyl salicylate, thymol, terpineol, verbenone, berberine, curcumin, caryophyllene oxide, nerolidol and geraniol.

The mixtures and combi-bars of the present invention can contain insect repellents, i.e. compounds active against insects (“repellents”): repellents are agents which are intended to prevent insects coming into contact with the skin and becoming active thereon. They drive away the insects and evaporate slowly. The most frequently used repellent is diethyl toluamide (DEET).

Natural repellents, such as aniseed oil, bergamot oil, cedarwood oil, citronella oil, citrus peel oils, eucalyptus oil, spruce needle oil, lavandin oil, lavender oil, Leptospermum petersonii oil, bay leaf oil, massoi oil, Mentha arvensis oil, nutmeg oil, clove leaf oil, clove flower oil, neroli oil, origanum oil, peppermint oil, pennyroyal oil, spike lavender oil, tagetes oil, tea tree oil, thyme oil, vetiver oil, cinnamon leaf oil and cinnamon bark oil or mixtures thereof; fragrances such as 6-decalactone, γ-decalactone, 6-dodecalactone, 7-dodecalactone, (E,Z)-2,6-nonadienal, δ-nonalactone, γ-nonalactone, δ-octalactone, δ-octalactone, α-phellandrene, δ-undecalactone, γ-undecalactone, 1,8-cineol, 1-phenyl-1,3-propanediol, 2-nonenal, 3,4-dihydrocoumarin, 3,8-p-menthanediol, 4a,5,6,7,8,8a-hexahydrocoumarin, 8-acetoxy-2-menthanone, benzyl benzoate, camphor, citronellol, coumarin, geraniol, linalool, octahydrocoumarin, piperitone, pulegone, hexylcinnamaldehyde (3-hexyl-3-phenyl-2-propenal), cinnamaldehyde, coniferyl aldehyde or mixtures thereof; synthetic repellents, such as N,N-diethyltoluamide (N,N-diethyl-3-methylbenzamide, DEET), bis-(dimethylthiocarbamoyl) disulphide (thiram), ethylenebis(dithiocarbamic acid) disodium salt (nabam), butoxypoly(propylene glycol), N-butylacetanilide, 2,3,4,5-bis(butyl-2-ene)tetrahydrofurfural, butyl 3,4-dihydro-2,2-dimethyl-4-oxo-2H-pyran-6-carboxylate, dibutyl adiptate, di-n-butyl succinate, 2-butyl-2-ethyl-1,3-propanediol, di-n-propylpyridine 2,5-dicarboxylate, isobutyl 2-(2-hydroxyethyl)piperidine-1-carboxylate, dibutyl phthalate, dimethyl phthalate, indalone and 2-ethyl-1,3-hexanediol or mixtures thereof can be used. Isobutyl 2-(2-hydroxyethyl)piperidine-1-carboxylate and N,N-diethyltoluamide or insect repellent mixtures containing these compounds are particularly preferred.

The mixtures and combi-bars of the present invention can contain deodorants and/or antiperspirants, i.e. active compounds or compounds mixtures having a deodorant and/or antiperspirant action. These include antiperspirants based on aluminium, zirconium or zinc salts, deodorants, bactericides or bacteriostatic deodorising substances, such as, for example, triclosan, hexachlorophene, triclocarban, contain alcohols and cationic substances, such as, for example, quaternary ammonium salts and odour absorbers, such as, for example, ® Grillocin (combination of zinc ricinoleate and various additives) or triethyl citrate, optionally in combination with an antioxidant, such as, for example, butylhydroxytoluene or ion exchange resins. In so-called antiperspirants, the formation of perspiration can be suppressed by astringents—predominantly aluminium salts, such as aluminium hydroxychloride (aluminium chlorohydrate).

The mixtures and combi-bars of the present invention can contain antioxidants suitable or customary for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. acarotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl, glyceryl and oligoglyceryl esters thereof and salts thereof, dilauryl thiodipropionate, distearyl thiodipropionate, thiodipropionic acid and derivatives thereof (esters, ethers, peptides, lipids, nucleotides, nucleosides and salts), and sulphoximine compounds (e.g. buthionine sulphoximines, homocysteine sulphoximine, buthionine sulphones, penta-, hexa- and heptathionine sulphoximine) in very low tolerated doses (e.g. pmol to μmol/kg), also (metal) chelating agents (e.g. α-hydroxy fatty acids, phytic acid, lactoferrin, α-hydroxy acids (e.g. citric acid, lactic acid, malic acid), humic acid, bile acid, bile extracts, tannins, bilirubin, biliverdin, EDTA, EGTA and derivatives thereof, folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, ferulic acid and derivatives thereof, caffeic acid and derivatives thereof, sinapic acid and derivatives thereof curcuminoids and derivatives thereof, retinoids, ursolic acid, levulinic acid, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof, (e.g. ZnO, ZnSO₄), selenium and derivatives thereof (e.g. selenomethionine), stilbenes and derivatives thereof (e.g. stilbene oxide, trans-stilbene oxide), and the derivatives (salts, esters, ethers, sugars, nucleotides, nucleosides, peptides and lipids) of said active compounds. Natural extracts, e.g. from green tea, algae, grape seeds, wheatgerms, rosemary, flavonoids, quercetin, phenolic benzylamines. Coenzymes, such as, for example, coenzyme Q10, plastoquinone, menaquinone, ubiquinols 1-10, ubiquinones 1-10 or derivatives of these substances are also suitable.

The mixtures and combi-bars of the present invention can contain moisture regulators. The moisture regulators (“moisturisers”) used are, for example, the following substances: sodium lactate, urea, alcohols, sorbitol, glycerol, propylene glycol, 1,2-pentane deiol, collagen, elastin or hyaluronic acid, diacyl adipates, petrolatum, ectoin, urocanic acid, lecithin, pantheol, phytantriol, lycopene, algae extract, ceramides, cholesterol, glycolipids, chitosan, chondroitin sulphate, polyamino acids and sugars, lanolin, lanolin esters, amino acids, alpha-hydroxy acids (e.g. citric acid, lactic acid, malic acid) and derivatives thereof, sugars (e.g. inositol), alpha-hydroxy fatty acids, phytosterols, triterpene acids, such as betulinic acid or ursolic acid, algae extracts.

The mixtures and combi-bars of the present invention can contain skin-lightening substances, such as, for example, 2,7-dinitroindazole, thiocitrulline, lactoferrin, humic acid, bile acid, bile extracts, bilirubin, biliverdin, arbutin, kojic acid, hydroquinone, resorcinol, flavonoids, retinoids, soya milk, serine protease inhibitors or lipoic acid.

The mixtures and combi-bars of the present invention can also contain skin-regenerating complexes (skin repair complexes), which are obtainable, for example, from inactivated and disintegrated cultures of bacteria of the Bifidus group.

The mixtures and combi-bars of the present invention can also contain self-tanning agents, such as dihydroxyacetone, glyceraldehyde, indole and derivatives thereof.

The mixtures and combi-bars of the present invention can contain hair-smoothing agents. Hair-smoothing agent as understood herein are substances which lead to the human or animal hair being smoothed. Suitable hair-smoothing agents are, for example, ammonium hydroxide, ammonium thioglycolate, calcium hydroxide and sodium hydroxyide.

The mixtures and combi-bars of the present invention can contain depilatory agents. Depilatory agents as understood herein are substances which lead to hair being removed from the skin.

Suitable depilatory agents are, for example, barium sulphide, magnesium thioglycolate, strontium sulphide, calcium sulphide, thiopropionic acid, strontium thioglycolate, calcium thioglycolate, potassium sulphide, thioglycerol, ethanolamine thioglycolate, potassium thioglycolate, thioglycolic acid, lithium sulphide, sodium sulphide, thiolactic acid, magnesium sulphide, ammonium thioglycolate and sodium thioglycolate.

The mixtures and combi-bars according to the present invention can also contain anti-dandruff active compounds, such as, for example, climbazole, ketoconazole or zinc pyrithione.

The mixtures and combi-bars of the present invention can also contain active compounds selected from the following group, oily or oil-soluble active compounds being preferred: acetylsalicylic acid, atropine, azulene, hydrocortisone and derivatives thereof e.g. hydrocortisone 17-valerate, vitamins, e.g. vitamin A and derivatives, ascorbic acid and derivatives thereof, vitamins of the B and D series, very beneficially vitamin B₁, vitamin B₁₂, niacinamide (nicotinamide), vitamin D₁, vitamin E (tocopherol) and derivatives thereof, vitamin F, panthenol, pantothenic acid, folic acid, and combinations thereof, but also bisabolol, unsaturated fatty acids, specifically the essential fatty acids (often also called vitamin F), in particular γ-linolenic acid, oleic acid, eicosapentaenoic acid, docosahexaenoic acid and derivatives thereof, chloramphenicol, caffeine, prostaglandins, thymol, camphor, extracts or other products of vegetable and animal origin, e.g. evening primrose oil, borage oil or currant seed oil, fish oils, cod-liver oil or also ceramides and ceramide-like compounds and so on, vitamin-based extracts: active compound compositions primarily with vitamin A, C, E, B₁, B₁₂, folic acid and biotin, amino acids and ferments, and compounds of the trace elements magnesium, silicon, phosphorus, calcium, manganese, iron or copper. Vitamins, such as, for example, vitamins A and E, can be incorporated to vitalise the skin.

The mixtures and combi-bars of the present invention can also comprise colorants. Colorants suitable for use in the mixtures and combi-bars can preferably be selected from the Symrise Dragocolor product range (www.symrise.com/en/fragrances/pdf/dictionary_of_colors.pdf). Particularly suitable colorants are water-soluble or water dispersible dyes which include Hansa Yellow G (C.I. 11680), Red (C.I. 12490), Orange II (C.I. 15510), Pyranine (C.I. 59040), Acid Green (C.I. 61570), Blue (C.I 61585) and combinations thereof. Particularly suitable pigments are dermatologically acceptable metal oxides, e.g. titanium dioxide.

The mixtures and combi-bars of the present invention may also comprise electrolytes, minerals or salts such as sodium chloride (typically up to 0.5 wt. %) and chelating agents such as EDTA or citric acid.

The mixture or combi-bar of the present invention may also comprise agents for adjusting a pH value or the ionic strength. Examples which may be mentioned are phosphoric acid and salts thereof, sodium acetate, acetic acid, citric acid and salts thereof, EDTA, disodium EDTA and tetrasodium EDTA.

In addition, the following ingredients and materials, for example, are possibly incorporated into the mixtures and combi-bars of the present invention: vegetable waxes and oils, such as, for example, cocoa butter, almond oil, avocado oil or jojoba oil for improving the feel of the skin.

Mixtures and combi-bars of the present invention may comprise abrasives customary in cosmetic and dermatological preparations. Natural or synthetic minerals which can have an abrading or abrasive action are, for example, dolomite, calcium carbonate, aragonite, feldspars, aluminium oxide, silicon dioxide, quartz, quarzite, gypsum, pumice, calcite, limestone, lime, artificial marble, marble, zirconium oxide, titanium dioxide, talc, sand, quartz sand, zirconium silicate, bentonites, precipitated chalk, magnesium carbonate, almond, peach and apricot kernel flour, wheatgerm flour, rice cornflour, olive kernel flour and walnut kernel flour. Pumice, calcite, limestone, lime, chalk, artificial or natural marble are particularly suitable. The hardness of the abrasive is preferably in the range from 1 to 4 on the Mohs' scale. The particle size is advantageously in the range 1 to 70, preferably in the range 1 to 60 and particularly preferentially in the range 1 to 50 micrometres. The total amount of abrasives (one or more) is preferably 0.5 to 10 wt. %, based on the total weight of the soap combi-bar.

Mixtures and combi-bars of the present invention may comprise skin conditioners, emollients, moisturizers, preservatives which are preferably selected form the group consisting of Dragoxat EH (Ethylhexyl Ethylhexanoate), Dragoderm (Glycerin, Wheat Gliadins), Hydrolite-5 (1,2-pentane diol), Symdiol 68 (a 1:1 (m/m) mixture of 1,2-hexane diol and 1,2-octane diol), 1,2-decane diol, PCL-Liquid (Cetearyl Ethylhexanoate, Isopropyl Myristate), alpha-bisabolol (Dragosantol 100), Dragocid Liquid (a mixture of phenoxyethanol, methylparaben, ethylparaben, butylparaben, propylparaben and isobutylparaben) and combinations thereof. The listed ingredients can be obtained from Symrise.

### Method for Producing Combi-Bar:

The steps of a method for producing a combi-bar floating in water at 20° C., and, preferably, other temperatures, according to the present invention, have been stated above.

Preferably, the fatty acids (component ii)) are melted at a temperature in the range of 70-90° C., preferably in the range of 70-80° C. The non-soap surfactants (component i)) are then dissolved in the molten fatty acids. The metal fatty acids soap (component iii)), preferably in the form of flakes or fine noodles, is added thereto and dissolved in the mixture of components i) and ii). The temperature may be increased to about 80-90° C., to keep the mixture in a molten state.

It has to be noted that the dissolving of component iii) (metal C₈ bis C₂₄ fatty acid soap) in the mixture becomes difficult and requires a higher processing temperature when the content of component iii) increases beyond 40% in the mixture.

Cautions stirring facilitates the mixing/dissolving processes. Prolonged vigorous mixing should be avoided.

Further ingredients (as stated above) may be incorporated into the mixture of compounds i), ii) and iii) prior to casting the mixture into moulds.

The cast moulds may be allowed to cool at room temperature, or cooling is accelerated by placing the moulds in a tray of cold water. The cast floating combi-bars can usually be removed from the moulds within 2 hours of casting.

It should be noted that the mixture of the present invention should be treated gently (in particular when the mixture is used for producing floating combi-bars). For instance, processes like shearing in a sigma mixer or processing through a crutcher or soap plodder should be avoided. Likewise, prolonged heating of the mixture to temperatures exceeding 90° C. should be avoided. A gentle treatment of the mixture is recommended in order to preserve the floatage of the mixture or the corresponding combi-bar. It was surprisingly found that a non-gentle treatment of a mixture according to the present invention in some cases leads to a mixture with an increased density and worsened floatage. The reason for these effects might be a disruption of the phase of free fatty acids (component ii)) and a more excessive emulsification of said free fatty acids by the surfactants (soap and non-soap, components i) and iii)). A suitable visual indicator for floatage, i.e. density, and probably the degree of emulsification of the fatty acids (component ii)) is the opacity/translucency of the heated mixture prior to the casting process. The more opaque (i.e. the less translucent) the heated mixture is, the higher is normally the floatage (i.e. the lower is the density).

It is preferred that the fatty acid phase is a continuous phrase encasing the soap and non-soap ingredients.

The following Examples illustrate the present invention.

### EXAMPLES 1-5

According to the procedure described above under the heading “method for producing combi-bar” five samples where prepared, according to Examples 1-5. Formulation details are stated in the following table:

| | Example | | | | |
|---|---|---|---|---|---|
| | 1 | 2 | 3 | 4 | 5 |
| Hostapon SCI-65 | 50% | 45% | 50% | 55% | 30% |
| (Sodium Cocoyl Isethionate, 65% | | | | | |
| and Stearic Acid, 35%), components | | | | | |
| i), ii) | | | | | |
| Edenor ST 1 MY | 25% | 25% | 35% | 34% | 42% |
| (Palmitic/Stearic Acid), component | | | | | |
| ii) | | | | | |
| Prisavon 9220 | 25% | 30% | 15% | 11% | 28% |
| (Palm/Palm Kernel Soap, 80:20), | | | | | |
| component iii) | | | | | |
| Total (wt. %) | 100% | 100% | 100% | 100% | 100% |

Combi-bars according to Examples 1-5 produced with the above amounts of components i), ii) and iii) were able to float in water at 20° C., although the content of air or other gas in the respective combi-bars was less than 2 vol. %.

### EXAMPLE 6

Using the ingredients stated in the following table a mixture was prepared for producing a combi-bar.

As component i) a combination of anionic and amphoteric surfactants is used. For producing a combi-bar according to the present invention the molten fatty acid (Edenor ST 1 MY, component ii)) is used to dissolve the soap and non-soap surfactants at a working temperature of 80-90° C. Perfume and titanium dioxide were added as further ingredients to the mixture of components i), ii) and iii) prior to casting in moulds.

The cast moulds are placed on a tray of water to accelerate the cooling process, and the bars are hardened and removed from the moulds after 1 hour. The resulting combi-bars floated when placed in water.

| | Supplier | wt. % | remarks |
|---|---|---|---|
| Hostapon SCI-65 | Clariant | 54 | 65% component i) 35% |
| (Sodium Cocoyl | | | |
| Isethionate, 65% | | | |
| and Stearic Acid, 35%) | | | component ii) |
| Tego Betain CKD | Goldschmidt | 1 | component i) |
| (Cocoamidopropyl | | | |
| Betaine, 82%) | | | |
| Edenor ST 1 MY | Cognis | 25 | component ii) |
| (Palmitic/Stearic Acid) | | | |
| Soap Base | | 18 | component iii) |
| Perfume | Symrise | 1.5 | component iv) |
| Titanium Dioxide | | 0.5 | component iv) |
| Total | | 100 | |

### EXAMPLE 7

A process of manufacture was carried out as described in Example 6, using the ingredients stated in the following table. The resulting combi-bars floated when placed in water.

| | Supplier | wt. % |
|---|---|---|
| ELFAN AT 84 | Akzo Nobel | 38 |
| (80% Sodium Cocoyl Isethionate, | | |
| 9% free acids, 2% water %), components | | |
| i), ii), and iv) | | |
| Prifrac 2942 | Uniqema | 43 |
| (Myristic Acid), component ii) | | |
| Soap Base, component iii) | | 18 |
| Perfume; component iv) | Symrise | 1 |
| Total | | 100 |

### EXAMPLE 8

A process was carried out as described in Example 6, using the ingredients stated in the following table.

| | Supplier | wt. % | remarks |
|---|---|---|---|
| Hostapon SCI-65 | Clariant | 54 | 65% component |
| (Sodium Cocoyl Isethionate, | | | i) |
| 65% and Stearic Acid, 35%) | | | 35% component |
| | | | ii) |
| Tego Betain CKD | Goldschmidt | 1 | component i) |
| (Cocamidopropyl Betaine, 82%) | | | |
| Edenor ST 1 MY | Cognis | 25 | component ii) |
| (Palmitic/Stearic Acid) | | | |
| Translucent Soap Base | | 18 | 11% component |
| | | | i) |
| | | | 15.8% component |
| | | | iii) |
| | | | 73.2% component |
| | | | iv), |
| | | | (32.63% water + |
| | | | 40.57% other |
| | | | compounds) |
| Perfume | | 1.5 | component iv) |
| Titanium Dioxide | | 0.5 | component iv) |
| Total | | 100 | |

The resulting combi-bars were opaque and floated when placed in water. The floating combi-bars had the following composition:
i) 38.08 wt. % of non-soap surfactants, component i)
ii) 43.9 wt. % of free C₁₄-C₁₈ fatty acids, component ii)
iii) 2.84 wt. % of metal C₈-C₂₄ fatty acid soaps, component iii)
iv) 15.18 wt. % further ingredients, component vi)
   the water content was about 5.88 wt. % and the glycerine content 4.5 wt. %. Polyethylene glycol (molecular weight 6000) as part of the translucent soap base was used to improve the hardness of the floating combi-bar.

The translucent soap base had a composition according to the following table:

| | Supplier | wt. % | remarks |
|---|---|---|---|
| Glycerine | | 25 | component |
| | | | iv) |
| Soft Water | | 7.63 | component |
| | | | iv) |
| Trilon B Powder | BASF | 0.25 | component |
| (Tetrasodium salt of EDTA) | | | iv) |
| Potassium salt of Kortacid | Akzo Nobel | 8.6 | component |
| 1499 (Myristic Acid | | | iii) |
| Potassium salt) | | | |
| Sodium salt of Kortacid | Akzo Nobel | 7.2 | component |
| PH 10 (Palmitic/Stearic | | | iii) |
| Acid Sodium salts) | | | |
| Butylated Hydroxytoluene | | 0.02 | component |
| (BHT) | | | iv) |
| Pluriol E 6000 | BASF | 14 | component |
| (Polyethylene Glycol, | | | iv) |
| M.W. 6000) | | | |
| Texapon NSO | Cognis | 35 | 30% component |
| (30% Sodium Laureth Sulfate, | | | i) |
| 70% water) | | | 70% component |
| | | | iv) |
| Plantacare 818 UP | Cognis | 1 | 50% component |
| (50% Coco-Glucoside, | | | i) |
| 50% water) | | | 50% component |
| | | | iv) |
| Lorina | Symrise | 1 | component |
| (Perfume) | | | iv) |
| Abil Quat 8842 | Goldschmidt | 0.2 | component |
| (Dimethiconol) | | | iv) |
| Glydant | Lonza | 0.1 | component |
| (DMDM Hydantoin) | | | iv) |
| Total | | 100 | |

### EXAMPLES 9 AND 10

In Examples 9 and 10 ingredients where used as stated in the following table:

| | | Example | |
|---|---|---|---|
| | | example 9 | example 10 |
| | Supplier | wt. % | wt. % |
| Hostapon SCI-65 | Clariant | 54 | 54 |
| (Sodium Cocoyl Isethionate, | | | |
| 65% and Stearic Acid, 35%) | | | |
| Tego Betain CKD | Goldschmidt | 1 | 1 |
| (Cocamidopropyl Betaine, 82%) | | | |
| Edenor ST 1 MY | Cognis | 25 | 25 |
| (Palmitic/Stearic Acid) | | | |
| Prisavon 9220 | Uniqema | 8 | 13 |
| (Palm/Palm Kernel Soap Base, | | | |
| 80:20) | | | |
| Soft Water | | 10 | 5 |
| Perfume | | 1.5 | 1.5 |
| Color (1%) | | 0.5 | 0.5 |
| Total | | 100 | 100 |

### EXAMPLE 9

10 wt. % of soft water were incorporated into the formulation, and the mixture attained a very high viscosity that is impractical to cast in moulds. However, the hardened mass still floated when placed in water.

### EXAMPLE 10

5 wt. % of soft water were incorporated into the formulation, and the mixture attained a high viscosity. Although the mixture can be cast into moulds, the combi-bars have a rough surface. The resulting soap combi-bars of the present invention floated when placed in water.

However, water can be added to the bar by using raw materials containing water.

### EXAMPLE 11

### Relation of Buoyancy to Method of Manufacture

### Experiment 1

Samples were cast at different temperatures to determine if heat-treatment and possibly heat-added emulsification of fatty acids by surfactants (soap and non-soap) played a role in the buoyancy of the samples.

The formulation used for samples A, B, C and D was as stated in Example 6.

Sample A was processed in accordance to the invention and cast at a temperature not exceeding 90° C. It is noted that the appearance of the mixture prior to the casting process was opaque.

Sample B was subjected to additional heat and cast at a temperature not exceeding 100° C. A thin, translucent layer could be seen in the mixture prior to the casting process.

Sample C was subjected to additional heating and cast at a temperature just exceeding 100° C.

It is noted that the translucent layer had increased prior to the casting process, in comparison with sample B.

Sample D was subjected to additional heating until sample D was a clear solution, at a temperature above 100° C. (approximately 105° C.)

The cast samples A, B, C and D where placed into a container of water and the following phenomenon was noted:
Sample A floats on the water.
Sample B is suspended in the water.
Samples C and D sink to the bottom of the container.

Details of the experiment are summarized in the following table:

| | A | B | C | D |
|---|---|---|---|---|
| Appearance of | Opaque | Slight translucency | Medium | Clear |
| mixture | | | translucency | |
| Temperature of | 80° C. | 90° C.-100° C. | >100° C. | >100° C. |
| casting | | | | |
| Buoyancy | Floats | Suspended | Sinks | Sinks |

### Experiment 2

A floating combi-bar is cut and a portion is melted down and recast at a temperature exceeding 90° C.

The recast sample had a reduced buoyancy when placed in water.

1. A method for producing a combi-bar that floats in water at 20° C., comprising the following steps:
a) providing a mixture comprising:
i) 20-50 wt % of one or more non-soap surfactants,
ii) 25-60 wt % of one or more free C₁₄-C₁₈ fatty acids,
iii) 2-40 wt % of one or more metal C₈-C₂₄ fatty acid soaps,
iv) 0-20 wt % of further ingredients, and said mixture comprises 0 to 10 wt % of water as a further ingredient,
b) heating said mixture to a temperature in the range of 70-90° C. to obtain a pourable melt,
c) casting or pouring said melt into a mold,
d) cooling said melt in said mold so that the melt solidifies, and
e) removing the resulting combi-bar from said mold,
wherein in the process no or only so little air or other gas is incorporated into the mixture or melt that the resulting combi-bar contains less than 2 vol % of air or other gas.

2. The method according to claim 1, wherein said mixture comprises:
i) 25-40 wt % of one or more non-soap surfactants,
ii) 35-55 wt % of one or more free C₁₄-C₁₈ fatty acids,
iii) 5-25 wt % of one or more metal C₈-C₂₄ fatty acid soaps,
iv) 2-15 wt % of further ingredients, and
said mixture is containing less than 6 wt % of water.

3. The method according to claim 1, wherein the free C₁₄-C₁₈ fatty acids of said mixture are saturated and/or the metal C₈-C₂₄ fatty acid soaps of said mixture are alkali metal C₈-C₂₄ fatty acid soaps.

4. The method according to claim 1, wherein the sum of the components i) to iii) of said mixture is at least 80 wt %.

5. The method according to claim 1, wherein the pourable melt is opaque when casted or poured into the mold.

6. The method according to claim 1, wherein a temperature of 90° C. is not exceeded.

7. A combi-bar that floats in water at 20° C., comprising a mixture,
a. said mixture comprises:
i) 20-50 wt % of one or more non-soap surfactants,
ii) 25-60 wt % of one or more free C₁₄-C₁₈ fatty acids,
iii) 2-40 wt % of one or more metal C₈-C₂₄ fatty acid soaps,
iv) 0-20 wt % of further ingredients, and
said mixture comprises 0 to 10 wt % of water as a further ingredient, and
b) said combi-bar comprises less than 2 vol % of air or other gas.

8. The combi-bar according to claim 7, comprising a mixture, wherein said mixture comprises:
i) 25-40 wt % of one or more non-soap surfactants,
ii) 35-55 wt % of one or more free C₁₄-C₁₈ fatty acids,
iii) 5-25 wt % of one or more metal C₈-C₂₄ fatty acid soaps,
iv) 2-15 wt % of further ingredients, and
said mixture is containing less than 6 wt % of water.

9. The combi-bar according to claim 8, wherein the free C₁₄-C₁₈ fatty acids of said mixture are saturated and/or the metal C₈-C₂₄ fatty acid soaps of said mixture are alkali metal C₈-C₂₄ fatty acid soaps.

10. The combi-bar according to claim 7, wherein the sum of the components i) to iii) of said mixture is at least 80 wt %

11. The method according to claim 1, wherein the mixture comprises one or more solid non-soap surfactants.

12. The method according to claim 1, wherein the mixture comprises 35-55 wt % of one or more free C₁₄-C₁₈ fatty acids.

13. The method according to claim 1, wherein the mixture comprises 5-25 wt % of one or more metal C₈-C₂₄ fatty acid soaps.

14. The method according to claim 1, wherein the mixture comprises 2-20 wt % of further ingredients.

15. The method according to claim 1, wherein said mixture comprises 6 to 10 wt % of water.

16. The combi-bar according to claim 7, wherein the mixture comprises 25-40 wt % of one or more non-soap surfactants.

17. The combi-bar according to claim 7 wherein the one or more non-soap surfactants is solid.

18. The combi-bar according to claim 7, wherein the mixture comprises 35-55 wt % of one or more free C₁₄-C₁₈ fatty acids.

19. The combi-bar according to claim 7, wherein the mixture comprises 5-25 wt % of one or more metal C₈-C₂₄ fatty acid soaps.
